(19) United States
(12) Patent Application Publication
(10) Pub. No.: US 2019/0380781 A1
(43) Pub. Date: Dec. 19, 2019

(54) AIRWAY MODEL GENERATION SYSTEM AND INTUBATION ASSISTANCE SYSTEM

(71) Applicants: JOHNFK MEDICAL INC., New Taipei City (TW); OSENSE TECHNOLOGY CO., LTD., Taipei City (TW)

(72) Inventors: Hung-Ya Tsai, Taipei City (TW); You-Kwang Wang, Taipei City (TW); Fei-Kai Syu, New Taipei City (TW)

(73) Assignees: JOHNFK MEDICAL INC., New Taipei City (TW); OSENSE TECHNOLOGY CO., LTD., Taipei City (TW)

(21) Appl. No.: 16/193,044

(22) Filed: Nov. 16, 2018

(30) Foreign Application Priority Data
Jun. 13, 2018 (CN) 201810608519.6

(51) Int. Cl. A61B 34/10; G06T 17/00; G06T 5/00; G06T 7/73; G06T 7/00; A61B 1/005; A61B 1/05; G06K 9/62; A61M 16/04; A61B 1/267

(52) U.S. Cl. CPC A61B 34/10; A61B 5/7264; G06T 5/002; G06T 7/75; G06T 7/0014; A61B 1/005; A61B 1/05; G06K 9/6214; A61M 16/0488; A61B 1/267; G06T 2210/41; G06T 2207/10068; G06T 2207/20081; G06T 2207/10028; G06T 17/00

(57) ABSTRACT

This application provides an airway model generation system, including an endoscope apparatus and a computer apparatus, to establish a three-dimensional model of an airway for a patient by using a simultaneous localization and mapping technology. This application further provides an intubation assistance system, to provide assistance to a medical worker during intubation treatment by using three-dimensional models of numerous patients and a machine learning technology.

### CROSS-REFERENCE TO RELATED APPLICATION

This non-provisional application claims priority under 35 U.S.C. § 119(a) to Patent Application No. 201810608519.6 filed in China, P.R.C. on Jun. 13, 2018, the entire contents of which are hereby incorporated by reference.

### BACKGROUND

### Technical Field

This application relates to an endoscope system, and in particular, to an airway model generation system and an intubation assistance system.

### Related Art

When a patient cannot spontaneously breathe during general anesthesia, emergency treatment, or the like, intubation treatment is usually performed on the patient. However, a medical worker always performs an intubation operation based on experience, and may accidentally injure the patient.

### SUMMARY

In view of this, this application provides an airway model generation system, to establish a three-dimensional model for an airway of a patient. In addition, an intubation assistance system is provided by using three-dimensional models of numerous patients and a machine learning technology, to provide assistance to a medical worker during intubation treatment.

The airway model generation system includes an endoscope apparatus and a computer apparatus. The endoscope apparatus includes a flexible hose, a camera module, and a communication module. The camera module is located at a front end of the flexible hose, to capture a plurality of airway images in a process that the flexible hose enters an airway. The communication module is coupled to the camera module, to send the plurality of airway images captured by the camera module. The computer apparatus is in communication connection with the communication module of the endoscope apparatus, to obtain the plurality of airway images sent by the communication module, and to establish a three-dimensional model of the airway by using a simultaneous localization and mapping (SLAM) technology based on the plurality of airway images.

The intubation assistance system includes an endoscope apparatus and a computer apparatus. The endoscope apparatus includes a flexible hose, a camera module, and a communication module. The camera module is located at a front end of the flexible hose, to capture a plurality of target airway images in a process that the flexible hose enters a target airway of a target patient. The communication module is coupled to the camera module, to send the plurality of target airway images captured by the camera module. The computer apparatus includes an input module, a storage module, a processing module, and an output module. The input module receives target patient data of the target patient. The storage module stores a patient database, where the patient database includes airway data and pathological data that correspond to each patient, and each airway data includes a plurality of airway images of an airway corresponding to the patient and a three-dimensional model of the airway. The processing module inputs the pathological data and the three-dimensional models of the patients to a first learning model. The first learning model provides first logic to evaluate a correlation between one or more eigenvalues in the pathological data and the three-dimensional model of the corresponding airway, and inputs the target patient data to the first learning model, to find a similar three-dimensional model from the three-dimensional models based on the first logic. The processing module further determines, based on the target airway images, that the front end of the flexible hose is located at a location in the similar three-dimensional model, to generate guidance information based on the location. The output module outputs the guidance information.

In conclusion, an embodiment of this application provides an airway model generation system, to establish a three-dimensional model of an airway for a patient during intubation. In addition, an embodiment of this application also provides an intubation assistance system, where a three-dimensional model of an airway of each patient and a corresponding airway image are documented and input to a learning model. By means of machine learning, a correlation between pathological data and the three-dimensional model of the airway is found, and a correlation between the airway image and the pathological data is found, to assist an intubation operation of a medical worker, and remind the medical worker of a possibly suffered disease.

### BRIEF DESCRIPTION OF THE DRAWINGS

FIG. 1 is a schematic architectural diagram of an airway model generation system and an intubation assistance system according to an embodiment of this application;

FIG. 2 is a schematic block diagram of an airway model generation system according to an embodiment of this application;

FIG. 3 is a flowchart of a method for generating an airway model according to an embodiment of this application;

FIG. 4 is a schematic block diagram of an airway model generation system according to another embodiment of this application;

FIG. 5 is a flowchart of a method for generating an airway model according to another embodiment of this application; and

FIG. 6 is a schematic operation diagram of an intubation assistance system according to an embodiment of this application.

### DETAILED DESCRIPTION

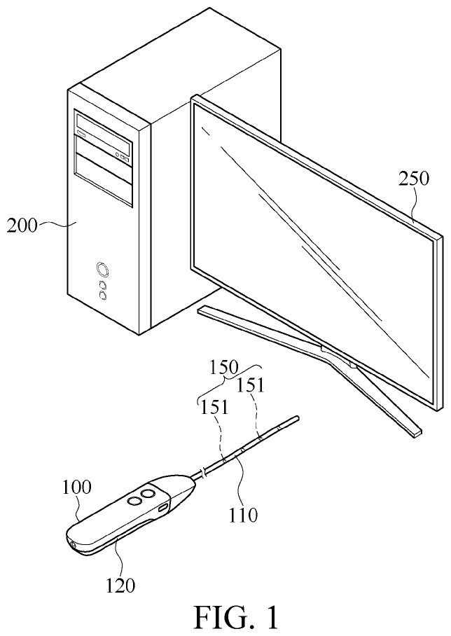

Referring to FIG. 1, FIG. 1 is a schematic architectural diagram of an airway model generation system and an intubation assistance system according to an embodiment of this application. The airway model generation system and the intubation assistance system include an endoscope apparatus 100 and a computer apparatus 200. The airway model generation system is first described below.

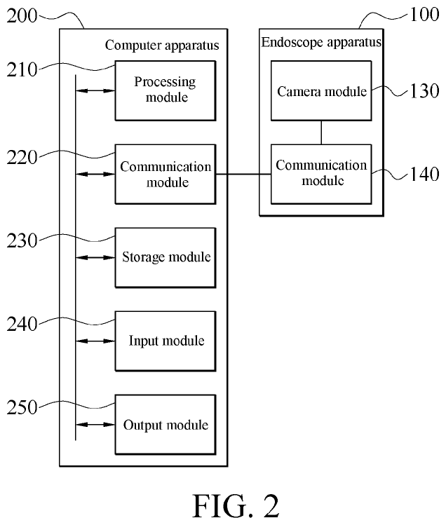

Referring to FIG. 1 and FIG. 2, FIG. 2 is a schematic block diagram of an airway model generation system according to an embodiment of this application. The endoscope apparatus 100 includes a flexible hose 110, a holding portion 120, a camera module 130, and a communication module 140. The flexible hose 110 is connected to the holding portion 120, so that a medical worker holds the holding portion 120 in hand and inserts the flexible hose 110 into an airway of a patient. The camera module 130 is disposed at a front end of the flexible hose 110, to capture an image in front of the flexible hose 110. Therefore, in a process that the flexible hose 110 enters the mouth of the patient and goes deep into the airway, airway images may be captured in a continuous or intermittent manner or by means of triggering. The camera module 130 may include one or more camera lenses. The camera lenses may be charge coupled devices (CCD) or complementary metal-oxide semiconductor (CMOS) image sensors. The communication module 140 may support a wired communication technology or a wireless communication technology. The wired communication technology may be, for example, low voltage differential signaling (LVDS) or Composite Video Broadcast Signal (CVBS). The wireless communication technology may be, for example, Wireless Fidelity (WiFi), Wi-Fi Display (WiDi), or Wireless Home Digital Interface (WHDI). The communication module 140 is coupled to the camera module 130, to transmit captured airway images to the computer apparatus 200.

The computer apparatus 200 includes a processing module 210 and a communication module 220. The communication module 220 supports a communication technology the same as that used by the communication module 140 of the endoscope apparatus 100, so that the communication module 220 is in communication connection with the communication module 140 of the endoscope apparatus 100, to obtain the airway images. The processing module 210 is coupled to the communication module 220, to establish a three-dimensional model of the airway by using a SLAM technology based on the airway images. The processing module 210 is processor having a computing capability, such as a central processing unit (CPU), a graphics processing unit (GPU), or a visual processing unit (VPU). The processing module 210 may include one or more of the foregoing processors.

In some embodiments, the computer apparatus 200 is a computing device.

In some embodiments, the computer apparatus 200 includes a plurality of same or different computing devices, for example, uses a distributed computing architecture or a computer cluster technology.

The computer apparatus 200 further includes a storage module 230, an input module 240, and an output module 250 that are coupled to the processing module 210. The storage module 230 is a non-transient storage medium, used to store the foregoing airway images. The output module 250 may be an image output apparatus, for example, one or more displays, used to display the airway images. The input module 240 may be a human-machine interface, and include a mouse, a keyboard, a touchscreen, and the like, so that the medical worker operates the computer apparatus 200.

In some embodiments, the endoscope apparatus 100 may further be provided with a display (not shown), to display the airway images captured by the camera module 130.

In some embodiments, if the endoscope apparatus 100 is provided with a display, the computer apparatus 200 may not be provided with the display.

In some embodiments, different from the foregoing endoscope apparatus 100 and the foregoing computer apparatus 200 that are two separable individuals, the endoscope apparatus 100 and the computer apparatus 200 are integrated in a same electronic device.

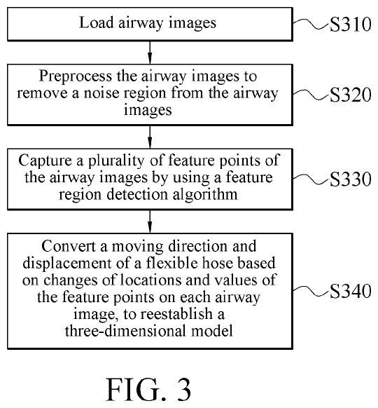

Referring to FIG. 3, FIG. 3 is a flowchart of a method for generating an airway model according to an embodiment of this application. The method is performed by the processing module 210, to implement the foregoing SLAM technology. First, the airway images stored in the storage module 230 are read and loaded (step S310). Next, the airway images are preprocessed to remove a noise region from the airway images (step S320). The noise region may be a region affecting image interpretation, for example, a mucous membrane or a blister. In step S330, a plurality of feature points of the airway images are captured by using a feature region detection algorithm. The feature region detection algorithm may be an algorithm such as a speed-up robust feature (SURF), a scale-invariant feature transform (SIFT), or an oriented BRIEF (ORB). Then, a moving direction and displacement of the flexible hose 110 may be converted based on changes of locations and values of the corresponding feature points on each airway image, to reestablish a three-dimensional model (step S340).

In some embodiments, images captured by a camera module 130 having two camera lenses may be used by the processing module 210 to implement a binocular vision SLAM algorithm, to reestablish the three-dimensional model.

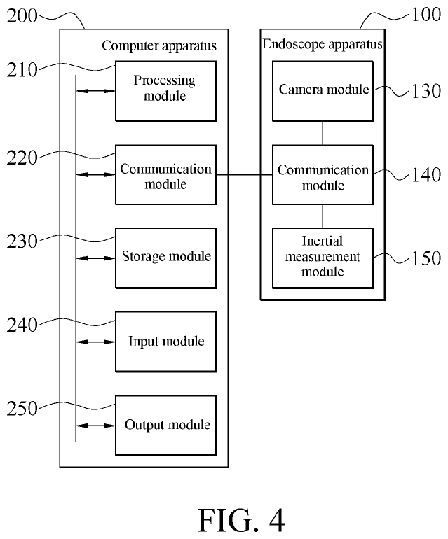

Referring to FIG. 4, FIG. 4 is a schematic block diagram of an airway model generation system according to another embodiment of this application. A difference from FIG. 2 lies in that an endoscope apparatus 100 in this embodiment further includes an inertial measurement module 150. The inertial measurement module 150 includes at least one inertial measurement unit 151, disposed on a flexible hose 110 (as shown in FIG. 1). The inertial measurement unit is used to obtain an inertial signal. For example, an accelerometer is used, so that a moving direction and an acceleration change of the flexible hose 110 can be learned. The inertial signal is transmitted to a computer apparatus 200 by using a communication module 140. Then, the computer apparatus 200 establishes, based on the inertial signal and airway images, a three-dimensional model of an airway by using a branch of the foregoing SLAM technology, that is, a visual-inertial odometry (VIO) technology.

In some embodiments, inertial measurement units 151 are evenly distributed in a long-axis direction of the flexible hose 110. In other words, on the flexible hose 110, the inertial measurement units 151 are disposed at intervals. In this way, bending deformation, a displacement direction, and displacement of each location of the flexible hose 110 can be learned based on inertial signals of the inertial measurement units 151.

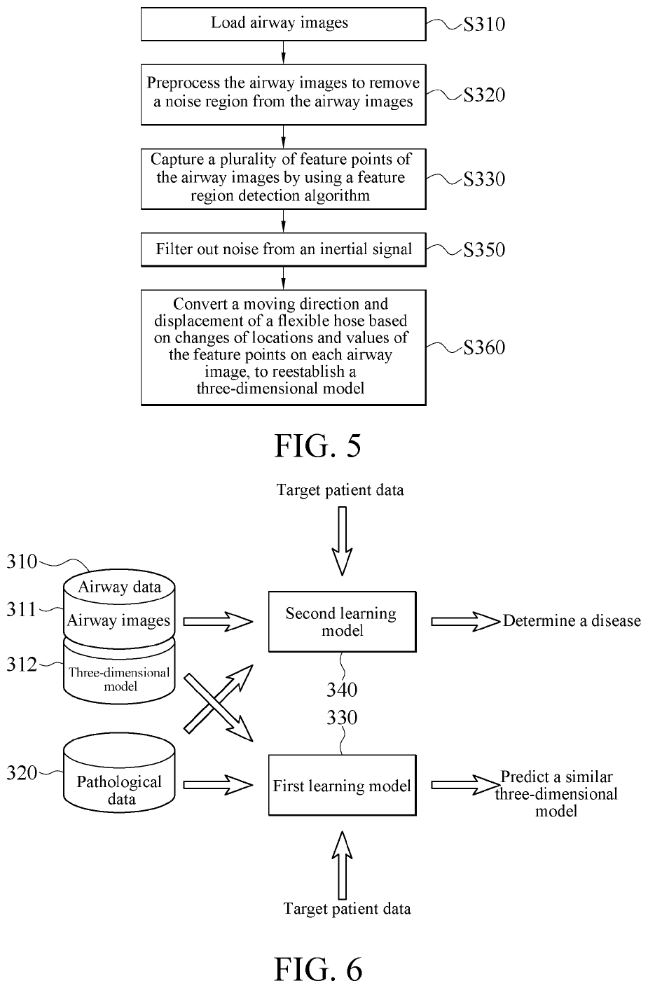

Referring to FIG. 5, FIG. 5 is a flowchart of a method for generating an airway model according to another embodiment of this application. A difference from FIG. 3 lies in that an endoscope apparatus 100 (as shown in FIG. 4) in this embodiment further includes an inertial measurement module 150. Therefore, after obtaining feature points according to step S310 to step S330, the computer apparatus 200 converts a moving direction and displacement of the flexible hose 110 based on changes of locations and values of the feature points on each airway image and an inertial signal, to reestablish a three-dimensional model (step S360). In addition, before step S360, the inertial signal may be preprocessed, to filter out noise from the inertial signal (step S350). Herein, step S350 is not limited to being performed between step S330 and step S360, and only needs to be performed before step S360. For example, a Kalman filter, a Gaussian filter, a particle filter, or the like may be used to filter out noise from the inertial signal.

In some embodiments, in step S320, that is, the airway images are preprocessed to remove a noise region from the airway images, the noise region is identified by means of machine learning so as to be removed. In other words, airway images of each patient may be input to a learning model, and the learning model may be selected from types such as supervised learning, unsupervised learning, semi-supervised learning, and reinforcement learning. For example, the learning model is a neural work, a random forest, a support vector machine (SVM), a decision tree, or a cluster. A correlation between a particular feature point and the noise region in the airway images are evaluated by using the learning model, to point out the noise region in the airway images.

Next, an intubation assistance system is described. The intubation assistance system is used to assist a medical worker in performing a correct operation when intubation is performed a current target patient, to avoid injuring the patient due to an incorrect operation. For hardware components of the intubation assistance system, refer to FIG. 1, FIG. 2, FIG. 4, and the foregoing descriptions, and details are not described herein.

Referring to FIG. 6, FIG. 6 is a schematic operation diagram of an intubation assistance system according to an embodiment of this application. It is particularly noted that a storage module 230 of a computer apparatus 200 can store a patient database. The patient database includes airway data 310 and pathological data 320 that correspond to each patient. The airway data 310 includes airway images 311 and three-dimensional model 312 of airways reestablished by using the foregoing method. The pathological data 320 is disease data, physical examination data, and the like of the patients. Before each intubation is performed, a medical worker enters target patient data (for example, basic data such as gender, a body height, or a weight and/or medical record data) of a current target patient by using the foregoing input module 240. The target patient data is added to the patient database. The data may be manually entered or input by using another method (for example, reading a file, reading a wafer, subscribing to an electronic medical record).

The processing module 210 inputs the pathological data 320 and the three-dimensional models 312 of the patients to a first learning model 330. The first learning model 330 may be selected from types such as supervised learning, unsupervised learning, semi-supervised learning, and reinforcement learning. For example, the first learning model is a neural work, a random forest, a support vector machine (SVM), a decision tree, or a cluster. The first learning model 330 provides first logic to evaluate a correlation between one or more eigenvalues in the pathological data 320 and the three-dimensional model 312 of the corresponding airway. The first logic is calculating, based on a relationship between values, weights, or the like of the one or more eigenvalues, probabilities of corresponding to the three-dimensional models 312 of airways of all or some of the patients. In some embodiments, a plurality of representative airway model samples may also be generated based on the three-dimensional models 312 of the patients. However, the first logic is calculating, based on a relationship between values, weights, or the like of the one or more eigenvalues, probabilities of corresponding to the airway model samples. For example, some eigenvalues represent a type of an airway in which difficult intubation easily occurs.

After the foregoing training, the processing module 210 inputs the target patient data to the first learning model 330, to find a similar three-dimensional model (that is, a model having a highest probability) from the three-dimensional models 312 based on the first logic. Then, when the medical worker performs intubation, the processing module 210 determines, by using the foregoing SLAM technology or the VIO technology based on airway images (referred to as target airway images below) of the target patient or in combination with the foregoing inertial signal, that a front end of a flexible hose 110 is located at a location in the similar three-dimensional model 312, to generate guidance information based on the location. For example, the guidance information may be guidance on a direction. The output module 250 may display the guidance information by using the foregoing display in a form of words or diagrams, and/or output the guidance information by combining another output manner, for example, a speaker, in another form such as voice.

In some embodiments, the processing module 210 further inputs the airway images 311 of the patients to a second learning model 340. The second learning model 340 may be selected from types such as supervised learning, unsupervised learning, semi-supervised learning, and reinforcement learning. For example, the second learning model is a neural work, a random forest, a support vector machine (SVM), a decision tree, or a cluster. The second learning model 340 provides second logic to evaluate a correlation between one or more eigenvalues in the airway images 311 and at least one disease in the corresponding pathological data 320. The second logic is calculating, based on a relationship between values, weights, or the like of the one or more eigenvalues in the airway images 311, a probability with which each corresponding disease may be suffered. After the training, the processing module 210 inputs the target airway images of the target patient to the second learning model, to evaluate, based on the second logic, a probability with which one or more diseases occur. The output module 250 may display a name of a possibly suffered disease by using the foregoing display in a form of words or diagrams, and/or output the name by combining another output manner, for example, a speaker, in another form such as voice.

In conclusion, embodiments of this application provide an airway model generation system, to establish a three-dimensional model of an airway for a patient during intubation. In addition, the embodiments of this application also provide an intubation assistance system, where a three-dimensional model of an airway of each patient and a corresponding airway image are documented and input to a learning model. By means of machine learning, a correlation between pathological data and the three-dimensional model of the airway is found, and a correlation between the airway image and the pathological data is found, to assist an intubation operation of a medical worker, and remind the medical worker of a possibly suffered disease.

What is claimed is:

1. An airway model generation system, comprising:
an endoscope apparatus, comprising:
a flexible hose;
a camera module, located at a front end of the flexible hose, to capture a plurality of airway images in a process that the flexible hose enters an airway; and
a communication module, coupled to the camera module, to send the plurality of airway images captured by the camera module; and
a computer apparatus, in communication connection with the communication module of the endoscope apparatus, to obtain the plurality of airway images sent by the communication module, and to establish a three-dimensional model of the airway by using a simultaneous localization and mapping (SLAM) technology based on the plurality of airway images.

2. The airway model generation system according to claim 1, wherein the endoscope apparatus further comprises an inertial measurement module, the inertial measurement module comprises at least one inertial measurement unit, to obtain at least one inertial signal, and the computer apparatus establishes the three-dimensional model of the airway based on the at least one inertial signal and the plurality of airway images and by using a visual-inertial odometry (VIO) technology.

3. The airway model generation system according to claim 2, wherein the at least one inertial measurement unit is evenly distributed in a long axis direction of the flexible hose.

4. The airway model generation system according to claim 2, wherein the computer apparatus further filters out noise from the at least one inertial signal.

5. The airway model generation system according to claim 1, wherein the computer apparatus comprises a processing module, and the processing module is configured to perform the following steps:
loading the plurality of airway images;
capturing a plurality of feature points of the plurality of airway images by using a feature region detection algorithm; and
converting a moving direction and displacement of the flexible hose based on changes of locations and values of the plurality of feature points of each airway image, to reestablish a three-dimensional model by using the SLAM technology.

6. The airway model generation system according to claim 5, wherein the processing module further preprocesses the plurality of airway images, to remove a noise region from the plurality of airway images.

7. An intubation assistance system, comprising:
an endoscope apparatus, comprising:
a flexible hose;
a camera module, located at a front end of the flexible hose, to capture a plurality of target airway images in a process that the flexible hose enters a target airway of a target patient; and
a communication module, coupled to the camera module, to send the plurality of target airway images captured by the camera module; and
a computer apparatus, comprising:
an input module, receiving target patient data of the target patient;
a storage module, storing a patient database, wherein the patient database comprises airway data and pathological data that correspond to each patient, and each airway data comprises a plurality of airway images of an airway corresponding to the patient and a three-dimensional model of the airway;
a processing module, inputting the pathological data of the patients and the three-dimensional models to a first learning model, wherein the first learning model provides first logic to evaluate a correlation between one or more eigenvalues in the pathological data and the three-dimensional model of the corresponding airway, and inputting the target patient data to the first learning model, to find a similar three-dimensional model from the three-dimensional models based on the first logic, wherein the processing module further determines, based on the target airway images, that the front end of the flexible hose is located at a location in the similar three-dimensional model, to generate guidance information based on the location; and
an output module, outputting the guidance information.

8. The intubation assistance system according to claim 7, wherein the processing module further inputs the plurality of airway images of the patients to a second learning model, the second learning model provides second logic to evaluate a correlation between one or more eigenvalues in the plurality of airway images and at least one disease in the corresponding pathological data, and inputs the plurality of target airway images of the target patient to the second learning model, to evaluate, based on the second logic, a probability with which the at least one disease occurs.
